(19) United States
(12) Patent Application Publication
(10) Pub. No.: US 2018/0177419 A1
(43) Pub. Date: Jun. 28, 2018

(54) METHOD AND DEVICE FOR PRESENTING ST EVENT IN ELECTROCARDIOGRAM

(71) Applicant: EDAN INSTRUMENTS, INC., Shenzhen, Guangdong (CN)

(72) Inventors: Dongxue Shen, Shenzhen, Guangdong (CN); Daxue Wei, Shenzhen, Guangdong (CN); Zaiyang Zhang, Shenzhen, Guangdong (CN)

(21) Appl. No.: 15/309,809

(22) PCT Filed: Sep. 13, 2016
(86) PCT No.: PCT/CN2016/098808

§ 371 Date: Nov. 9, 2016

(51) Int. Cl. A61B 5/044; A61B 5/0472; A61B 5/04; G16H 15/00

(52) U.S. Cl. CPC A61B 5/044; G16H 15/00; A61B 5/04012; A61B 5/0472

(57) ABSTRACT

A method for presenting an ST event in an electrocardiogram (ECG) comprises: drawing an amplitude trend graph of an ST segment in the ECG; detecting an ST event in the amplitude trend graph of the ST segment; and marking the detected ST event in the amplitude trend graph of the ST segment. In the present invention, the ST event is automatically detected and marked in the amplitude trend graph of the ST segment, so that the ST events are presented more intuitively, and the user can intuitively and quickly locate the ST event in the amplitude trend graph of the ST segment and quickly know a raised or depressed situation of the ST event; the time of subsequent analysis and summary can be effectively reduced, and a blood supply state of the heart of the subject can be timely and quickly known.

### FIELD OF THE INVENTION

The present invention relates to the technical field of biomedical signal processing, and more particularly to a method and device for presenting an ST event in an electrocardiogram.

### BACKGROUND OF THE INVENTION

An ST segment of an electrocardiogram (ECG) is used to represent ability of a heart to supply blood. The ST segment of the ECG corresponds to a second phase of an action potential, and the second phase of the action potential is also called as a plateau phase. Under normal circumstances, the ST segment is an equipotential line, but many physiological factors or pathological factors will break the balance, cause the ST segment up or down, and result in waveform abnormalities of the ST segment. Generation of the waveform abnormalities of the ST segment is hereinafter referred to as occurrence of an ST event. The physiological factors leading to the ST events are usually related to autonomic nervous excitability. And the most important factors in the pathological factors include myocardial ischemia, ventricular hypertrophy, ventricular conduction abnormalities, pericarditis, electrolyte disorders, drug effects, and so on. A user needs to further determine an observation conclusion according to changes of the ST segment in the ECG, therefore, the way to present the ST event in the ECG becomes particularly important.

In the ECG, the ST segment begins after QRS complex, and usually begins after an S-wave of the QRS complex and ends at front of a T-wave. The end of the S-wave is also called a J-point. At present, when an ECG signal is output, the method for presenting the ST events is relatively simple, which may result in limited intuition and effectiveness of observation and analysis for the ST events, and further lead to that an accuracy of analysis result is lowered.

### SUMMARY OF THE INVENTION

In view of the above, the embodiments of the present invention provide a method and device for presenting an ST event in an electrocardiogram (ECG), which aim at solve the problems in the prior art that when an ECG signal is output, the method for presenting the ST events is relatively simple, which may result in limited intuition and effectiveness of observation and analysis for the ST events.

In a first aspect, a method for presenting an ST event in an electrocardiogram (ECG) is provided, wherein the method comprises:

drawing an amplitude trend graph of an ST segment in the ECG;

detecting an ST event in the amplitude trend graph of the ST segment; and

marking the detected ST event in the amplitude trend graph of the ST segment.

As a first possible implementation of the first aspect, drawing an amplitude trend graph of an ST segment in the ECG comprises:

performing a QRS complex detection for ECG data collected from each lead, and obtaining characteristic points of the QRS complex by the detection;

determining the ST segment according to the characteristic points of the QRS complex; and

drawing the amplitude trend graph of the ST segment according to an amplitude of the ST segment.

As a second possible implementation of the first aspect, drawing an amplitude trend graph of an ST segment in the ECG comprises:

dividing an amplitude value range of the ST segment into a plurality of numerical intervals, and setting a corresponding drawing color for each of the numerical intervals;

for any point in time of the amplitude trend graph of the ST segment, determining a drawing color of an amplitude value corresponding to the point in time according to a numerical interval where the amplitude value corresponding to the point in time lies; and

drawing an amplitude value for each point in time of the amplitude trend graph of the ST segment, according to the determined drawing color.

As a third possible implementation of the first aspect, marking the detected ST event in the amplitude trend graph of the ST segment comprises:

dividing an amplitude value range of the ST segment in a period of time corresponding to the ST event into a plurality of numerical intervals, and setting a corresponding mark format for each of the numerical intervals; and

for any ST event, determining a mark format corresponding to the ST event according to a numerical interval where an amplitude value of the ST event lies, and marking the ST event.

As a fourth possible implementation of the first aspect, the method further comprises:

when it is detected that a mark of any ST event in the amplitude trend graph of the ST segment is triggered, displaying information of the ST event in a display area of an ST event list which is displayed in the same screen as the amplitude trend graph of the ST segment, and performing a display content jumping in an ECG waveform area which is displayed in the same screen as the amplitude trend graph of the ST segment, thereby displaying heartbeat data at the same time as a start position of the ST event in the ECG waveform area.

In combination with the fourth possible implementation of the first aspect, in a fifth possible implementation, the method further comprises:

if a selection event occurring in any one of the amplitude trend graph of the ST segment, the display area of the ST event list, and the waveform area of the ECG is detected, the other two of the amplitude trend graph of the ST segment, the display area of the ST event list, and the waveform area of the ECG simultaneously jumping to the display content at the same point in time as the point in time pointed by the selection event.

In combination with the fourth possible implementation of the first aspect, in a sixth possible implementation, the method further comprises:

if an editing operation for an ST event occurring in any one of the amplitude trend graph of the ST segment, the display area of the ST event list, and the waveform area of the ECG is detected, information of the ST event being synchronously updated according to the editing operation in the other two of the amplitude trend graph of the ST segment, the display area of the ST event list, and the waveform area of the ECG.

In a second aspect, a device for presenting an ST event in an ECG is provided, wherein the device comprises:

a drawing unit, configured for drawing an amplitude trend graph of an ST segment in the ECG;

a detection unit, configured for detecting an ST event in the amplitude trend graph of the ST segment; and

a mark unit, configured for marking the detected ST event in the amplitude trend graph of the ST segment.

As a first possible implementation of the second aspect, the drawing unit comprises:

a detection sub-unit, configured for performing a QRS complex detection for ECG data collected from each lead, and obtaining characteristic points of the QRS complex by the detection;

a first determining sub-unit, configured for determining the ST segment according to the characteristic points of the QRS complex; and

a first drawing sub-unit, configured for drawing the amplitude trend graph of the ST segment according to an amplitude of the ST segment.

As a first possible implementation of the second aspect, the device further comprises:

a display unit, configured for displaying information of the ST event in a display area of an ST event list which is displayed in the same screen as the amplitude trend graph of the ST segment, and performing a display content jumping in an ECG waveform area which is displayed in the same screen as the amplitude trend graph of the ST segment, thereby displaying heartbeat data at the same time as a start position of the ST event in the ECG waveform area, when it is detected that a mark of any ST event in the amplitude trend graph of the ST segment is triggered.

In combination with the second possible implementation of the second aspect, the device further comprises:

a linkage unit, configured for making the other two of the amplitude trend graph of the ST segment, the display area of the ST event list, and the waveform area of the ECG simultaneously jump to the display content at the same point in time as the point in time pointed by an selection event, if the selection event occurring in any one of the amplitude trend graph of the ST segment, the display area of the ST event list, and the waveform area of the ECG is detected.

In a third aspect, a device for presenting an ST event in an ECG is provided, wherein the device comprises a processor, a memory, and a bus;

the processor and the memory communicated with each other through the bus;

the memory, configured for storing programs; and

the processor configured for executing the programs stored in the memory, when the programs executed, configured for:

drawing an amplitude trend graph of an ST segment in the ECG;

detecting an ST event in the amplitude trend graph of the ST segment; and

marking the detected ST event in the amplitude trend graph of the ST segment.

As a first possible implementation of the third aspect, the processor is specifically configured for: performing a QRS complex detection for ECG data collected from each lead, and obtaining characteristic points of the QRS complex by the detection;

determining the ST segment according to the characteristic points of the QRS complex; and

drawing the amplitude trend graph of the ST segment according to an amplitude of the ST segment.

As a second possible implementation of the third aspect, the processor is specifically configured for:

dividing an amplitude value range of the ST segment into a plurality of numerical intervals, and setting a corresponding drawing color for each of the numerical intervals:

for any point in time of the amplitude trend graph of the ST segment, determining a drawing color of an amplitude value corresponding to the point in time according to a numerical interval where the amplitude value corresponding to the point in time lies; and

drawing an amplitude value for each point in time of the amplitude trend graph of the ST segment, according to the determined drawing color.

As a third possible implementation of the third aspect, the processor is specifically configured for:

dividing an amplitude value range of the ST segment in a period of time corresponding to the ST event into a plurality of numerical intervals, and setting a corresponding mark format for each of the numerical intervals; and

for any ST event, determining a mark format corresponding to the ST event according to a numerical interval where an amplitude value of the ST event lies, and marking the ST event.

As a fourth possible implementation of the third aspect, the processor is specifically configured for:

when it is detected that a mark of any ST event in the amplitude trend graph of the ST segment is triggered, displaying information of the ST event in a display area of an ST event list which is displayed in the same screen as the amplitude trend graph of the ST segment, and performing a display content jumping in an ECG waveform area which is displayed in the same screen as the amplitude trend graph of the ST segment, thereby displaying heartbeat data at the same time as a start position of the ST event in the ECG waveform area.

In combination with the fourth possible implementation of the third aspect, in a fifth possible implementation, the processor is specifically configured for:

if a selection event occurring in any one of the amplitude trend graph of the ST segment, the display area of the ST event list, and the waveform area of the ECG is detected, the other two of the amplitude trend graph of the ST segment, the display area of the ST event list, and the waveform area of the ECG simultaneously jumping to the display content at the same point in time as the point in time pointed by the selection event.

In combination with the fourth possible implementation of the third aspect, in a sixth possible implementation, the processor is specifically configured for:

if an editing operation for an ST event occurring in any one of the amplitude trend graph of the ST segment, the display area of the ST event list, and the waveform area of the ECG is detected, information of the ST event being synchronously updated according to the editing operation in the other two of the amplitude trend graph of the ST segment, the display area of the ST event list, and the waveform area of the ECG.

In the embodiments of the present invention, the ST event is automatically detected and marked in the amplitude trend graph of the ST segment, so that the ST events are presented more intuitively, and the user can intuitively and quickly locate the ST event in the amplitude trend graph of the ST segment and quickly know a raised or depressed situation of the ST event; the time of subsequent analysis and summary can be effectively reduced, and a blood supply state of the heart of the subject can be timely and quickly known.

### BRIEF DESCRIPTION OF THE DRAWINGS

In order to explain the technical solutions of the present invention clearly, a brief introduction regarding the accompanying drawings that need to be used for describing the embodiments or the prior art is given below; it is obvious that the accompanying drawings described as follows are only some embodiments of the present invention, for those skilled in the art, some other drawings can also be obtained according to the current drawings on the premise of paying no creative labor.

FIG. 1 is an implementation flow chart of a method for presenting an ST event in an electrocardiogram (ECG) provided by an embodiment of the present invention.

FIG. 2 is a specific implementation flow chart of a step S101 of the method for presenting an ST event in an ECG provided by an embodiment of the present invention.

FIG. 3 is a specific implementation flow chart of a step S101 of a method for presenting an ST event in an ECG provided by another embodiment of the present invention.

FIG. 4 is a specific implementation flow chart of a step S103 of the method for presenting an ST event in an ECG provided by an embodiment of the present invention.

FIG. 5 is an implementation flow chart of a method for presenting an ST event in an ECG provided by another embodiment of the present invention.

FIG. 6 is an implementation flow chart of a method for presenting an ST event in an ECG provided by another embodiment of the present invention.

FIG. 7 is an exemplary interface view of which a screen displays an amplitude trend graph of an ST segment, a display area of an ST event list, and a waveform area of the ECG simultaneously provided by an embodiment of the present invention.

FIG. 8 is an exemplary interface view of editing an ST event in an amplitude trend graph of an ST segment provided by an embodiment of the present invention.

FIG. 9 is an exemplary interface view of editing an ST event in a display area of an ST event list provided by an embodiment of the present invention.

FIG. 10 is an exemplary interface view of editing an ST event in a waveform area provided by an embodiment of the present invention.

FIG. 11 is a structural block diagram of a device for presenting an ST event in an ECG provided by an embodiment of the present invention.

FIG. 12 is a block diagram of a hardware architecture of a device for presenting an ST event in an ECG provided by an embodiment of the present invention.

### DETAILED DESCRIPTION OF THE PREFERRED EMBODIMENT

In the following description, for purposes of explanation and not limitation, specific details, such as specific system structures, techniques, are set forth in order to provide a thorough understanding of the embodiments of the present invention. However, it will be apparent to those skilled in the art that the present invention may be practiced in other embodiments without these specific details. In other instances, detailed descriptions of well-known systems, devices, circuits, and methods are omitted so as to avoid unnecessary details from obscuring the description of the present invention.

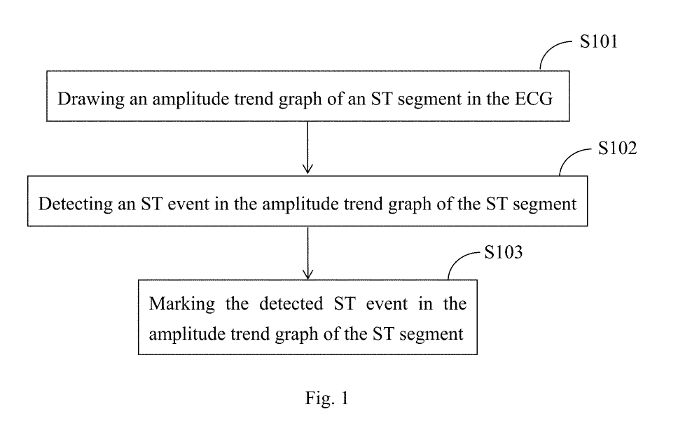

FIG. 1 illustrates an implementation flow chart of a method for presenting an ST event in an electrocardiogram (ECG) provided by an embodiment of the present invention, which is described in detail as follows:

In a step S101, drawing an amplitude trend graph of an ST segment in the ECG.

Before the step S101 being performed, it is necessary to import ECG data from an collection terminal, the ECG data may be data obtained after the collection terminal continuously collecting for 24 hours. Because the ECG data collected by the collection terminal is too much, it can not analyze the ECG data in real time during the collection process, and the ECG data is needed to import into an ECG analysis system for further analysis. In the import process of the ECG data, the adopted import methods comprise, but are not limited to, a memory card import, a universal serial bus (USB) data cable import, a short-range wireless communication (such as Bluetooth) way import or based on a Wireless Fidelity (WiFi) network to transmit the ECG data from the collection terminal to the ECG analysis system, and the like.

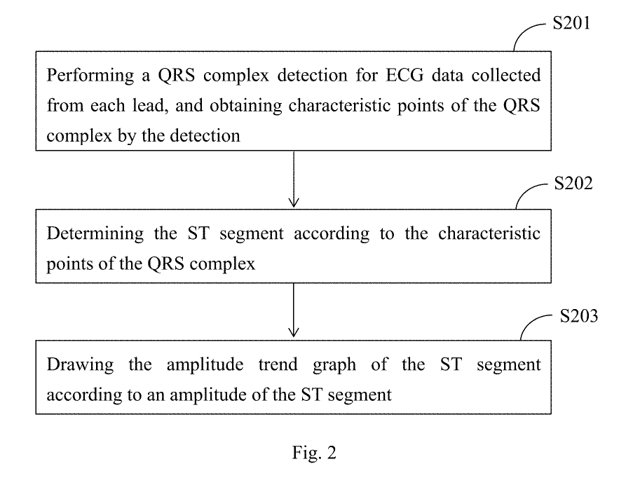

A specific implementation of the step S101 is shown in FIG. 2.

In a Step S201, performing a QRS complex detection for ECG data collected from each lead, and obtaining characteristic points of the QRS complex by the detection.

Generally, the ECG data is collected by a plurality of leads, therefore, performing the QRS complex detection for the ECG data collected from each lead can be implemented by detection methods such as slope-threshold arithmetic, tangential arithmetic and so on, thereby determining equipotential points, J-point, ST-point, and other characteristic points; alternatively, each of the characteristic points may be determined by the manually setting of the user.

In Step S202, determining the ST segment according to the characteristic points of the QRS complex.

After obtaining the characteristic points of the QRS complex, an end of an S wave and a starting position of a T wave are determined, and a waveform between the end of the S wave and the starting position of the T wave is determined as the ST segment.

In a step S203, drawing the amplitude trend graph of the ST segment according to an amplitude of the ST segment.

In one embodiment of the present invention, an abscissa of the amplitude trend graph of the ST segment is time, and an ordinate of the amplitude trend graph of the ST segment is the amplitude of the ST segment. The amplitude trend graph of the ST segment can reflect a situation that an ST wave of an ECG signal of the subject changes with time.

Preferably, after finishing drawing the amplitude trend graph of the ST segment, as an embodiment of the present invention, when it is detected that any occurrence time of the amplitude trend graph of the ST segment is triggered, ST segment information of the occurrence time is identified by an icon on the amplitude trend graph of the ST segment. Wherein, the occurrence time may be any one of the occurrence times indicated on the ordinate of the amplitude trend graph of the ST segment. The triggering methods of the occurrence time comprise, but are not limited to: clicking any position in the amplitude trend graph of the ST segment, and detecting an occurrence time corresponding to the position, so as to lead the occurrence time triggered; or manually inputting any one of the occurrence times in a range of the abscissa of the amplitude trend graph of the ST segment, so as to lead the occurrence time triggered. After any occurrence time being triggered, “the ST segment information of the occurrence time is identified by an icon” comprises, but is not limited to: a time value of the occurrence time, a magnitude value of the ST segment corresponding to the occurrence time, or lead information of the occurrence time. A shape of the icon is not limited here, and it can be a shape that can play a significant role in marking, such as a small red flag; in this way, after the occurrence time being triggered, the user can intuitively and quickly check an ST segment information corresponding to the occurrence time, cumbersome calculation process of the ST segment information is avoided, and efficiency of processing information is improved.

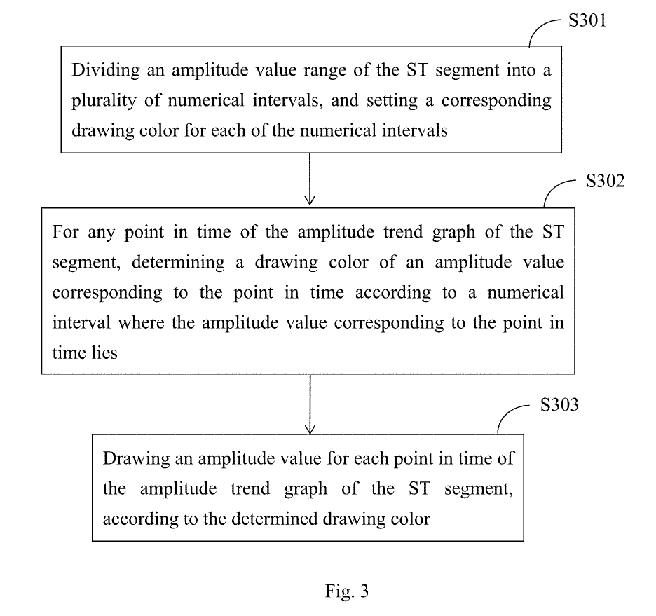

A preferred embodiment of the step S101 is shown in FIG. 3.

In a step S301, dividing an amplitude value range of the ST segment into a plurality of numerical intervals, and setting a corresponding drawing color for each of the numerical intervals.

In a step S302, for any point in time of the amplitude trend graph of the ST segment, determining a drawing color of an amplitude value corresponding to the point in time according to a numerical interval where the amplitude value corresponding to the point in time lies.

In step S303, drawing an amplitude value for each point in time of the amplitude trend graph of the ST segment, according to the determined drawing color.

In the embodiment corresponding to FIG. 3, the amplitude trend graph of the ST segment is drawn with different colors, which can intuitively display an offset direction, a value of magnitude, a duration of a magnitude of the ST segment, and so on, so that the ST segment can be presented more intuitively.

On the basis of the embodiment corresponding to FIG. 3, more particularly, the amplitude value range of the ST segment may be divided into a positive interval and a negative interval, and the positive interval and the negative interval may be drawn with different colors. Therefore, up or down situations of the ST segment can be displayed intuitively, which helps the user efficiently combine the up or down situations of the ST segment to make further analysis.

In a step S102, detecting an ST event in the amplitude trend graph of the ST segment.

In step S103, marking the detected ST event in the amplitude trend graph of the ST segment.

In the ST segment of the ECG, each ST event is represented as a waveform with its ST event characteristics. Generally speaking, if an amplitude value of the ST segment exceeds a preset value range, for example, the amplitude value of the ST segment is greater than a positive amplitude threshold or less than a negative amplitude threshold, that is, the ST segment is raised or depressed, an ST event can be identified. Therefore, as long as all of amplitude values of the amplitude trend graph of the ST segment are detected, the detection of the ST events is completed. After the ST events being detected, the ST events are marked. In one embodiment, mark methods for the ST events comprise, but are not limited to, marking with different colors, or adding shadows to the waveform for marking. Moreover, mark methods for the ST events can also comprise distinguishing the raised ST segment and the depressed ST segment, for example, marking the raised ST segment and the depressed ST segment with different colors.

A preferred embodiment of marking the ST event is shown in FIG. 4.

In step S401, dividing an amplitude value range of the ST segment in a period of time corresponding to the ST event into a plurality of numerical intervals, and setting a corresponding mark format for each of the numerical intervals.

In step S402, for any ST event, determining a mark format corresponding to the ST event according to a numerical interval where an amplitude value of the ST event lies, and marking the ST event.

Based on the embodiment corresponding to FIG. 4, it is possible to distinguish different types of the ST events by mark. For example, if the ST events are marked with colors, when an amplitude value range of the ST segment in a period of time corresponding to the ST event exceeds a positive ST event amplitude threshold or a negative ST event amplitude threshold, a color block for marking the ST event will be rendered in another display mode, for example, a height of the color block is higher than heights of color blocks for marking ordinary ST events, a color of the color block is darker than colors of color blocks for marking ordinary ST events, and so on. In this way, some ST events that require significant attention can be prominently marked to indicate the importance of the ST event to an analysis conclusion, to help the user to analyze the ST events and obtain a more accurate conclusion. For example, an ST event may be over-raised or over-depressed, and the user may be prompted by a significant mark to combine the ST event with other physiological parameters and thereby determine a blood supply state of ventricular muscle of the subject during a period of time in which the ST event continues.

In the embodiments of the present invention, the ST event is automatically detected and marked in the amplitude trend graph of the ST segment, so that the ST events are presented more intuitively, and the user can quickly obtain an analysis conclusion from the amplitude trend graph of the ST segment. Particularly, a length of the ST event can visually reflect a duration of the ST event, and the duration of the ST event is an objective reflection of the subject's body condition, moreover, the ST segment reflects blood supply state of a heart of the subject. For a dynamic ECG, the ECG data of the subject is collected continually for at least 24 to 72 hours, so that amount of the ECG data is too large, and to select the ST events from the amplitude trend graph of the ST segment is actually very difficult. Therefore, in the embodiments of the present invention, the ST event is automatically detected and marked in the amplitude trend graph of the ST segment, so that the ST events are presented more intuitively, and the user can intuitively and quickly locate the ST event in the amplitude trend graph of the ST segment and quickly know a raised or depressed situation of the ST event; the time of subsequent analysis and summary can be effectively reduced, and a blood supply state of the heart of the subject can be timely and quickly known.

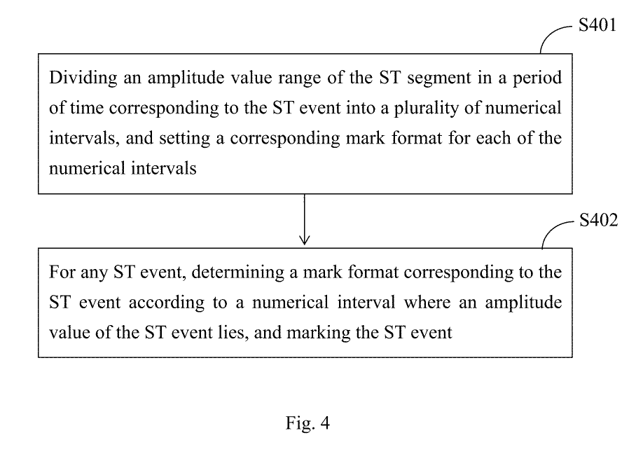

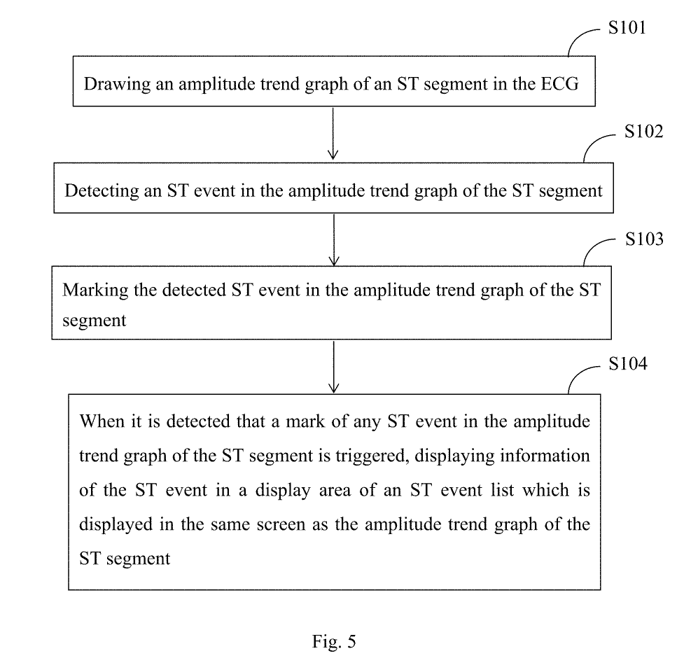

As an embodiment of the present invention, on the basis of the embodiment corresponding to FIG. 1, as shown in FIG. 5, after the step S103, the method further comprises:

In step S104, when it is detected that a mark of any ST event in the amplitude trend graph of the ST segment is triggered, displaying information of the ST event in a display area of an ST event list which is displayed in the same screen as the amplitude trend graph of the ST segment.

As mentioned above, the ST event reflects blood supply state of the heart of the subject. In the amplitude trend graph of the ST segment, a waveform change of the ST segment can be reflected only in a chronological order, however, the display area of the ST event list can show all kinds of information related to the ST event. The information comprises, but is not limited to, situation of the ST event (being raised or being depressed), occurrence time, duration, ST amplitude, leads to the ST event, heart rate before the time of the ST event happened, an average heart rate during the duration of the ST event, the fastest heart rate during the duration of the ST event, and so on. In the display area of the ST event, the displayed kinds of the information can be selected by the user. The user can flexibly select the kind of the information and a display order of the various kinds of information, which are displayed in the display area of the ST event, according to actual needs, and thereby flexibly customize the displayed information.

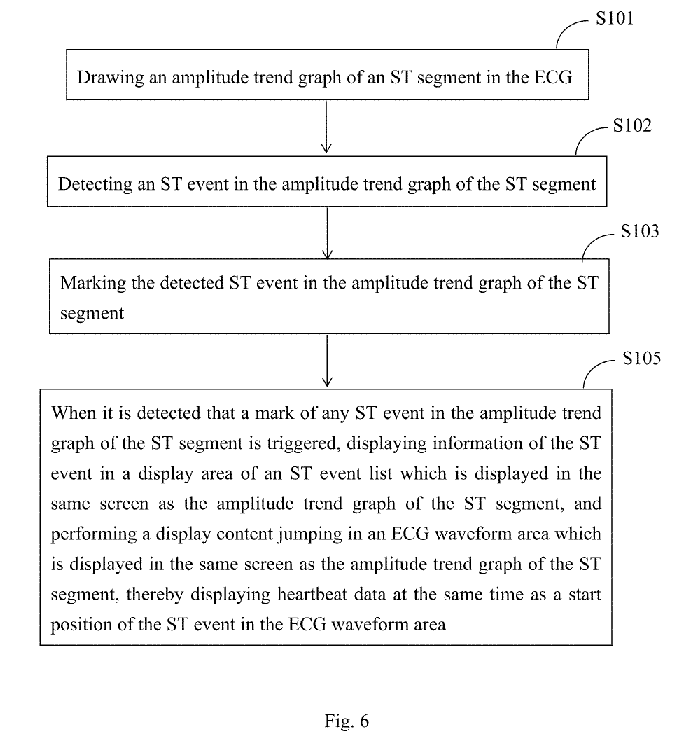

As an embodiment of the present invention, on the basis of the embodiment corresponding to FIG. 1, as shown in FIG. 6, after the step S103, the method further comprises:

In step S105, when it is detected that a mark of any ST event in the amplitude trend graph of the ST segment is triggered, performing a display content jumping in an ECG waveform area which is displayed in the same screen as the amplitude trend graph of the ST segment, thereby displaying heartbeat data at the same time as a start position of the ST event in the ECG waveform area.

The heartbeat is an ECG waveform of a heart beating once. The ECG is based on the same time axis, and all of the collected ECG data is displayed. Therefore, when it is detected that a mark of the ST event is triggered, on the basis of a point in time of the start position of the ST event, the same point in time in the waveform area of the ECG can be located, so that displaying the heartbeat data at the point in time is completed, and the user can obtain a specific situation of the ECG data at the occurrence of the ST event in all aspects.

On the basis of the embodiment corresponding to FIG. 6, while the display content jump is performed in the waveform area of the ECG, the heartbeat data at the same time as the start position of the ST event is marked in the waveform area of the ECG. Further, the time axis may be moved, so that the heartbeat data can be located at a center of the waveform area of the ECG. All of measures described above are designed to enable the user to locate the heartbeat data more quickly. Methods for marking the heartbeat data comprise, but are not limited to, the heartbeat data being marked with a rectangular block, a colored block, an arrow, a bold waveform, and the like.

When the heartbeat data is marked with a block, further, basic information of the ST event is displayed in an adjacent area of the block, and the basic information comprises an event type of the ST event or leads of occurrence of the ST event, and so on.

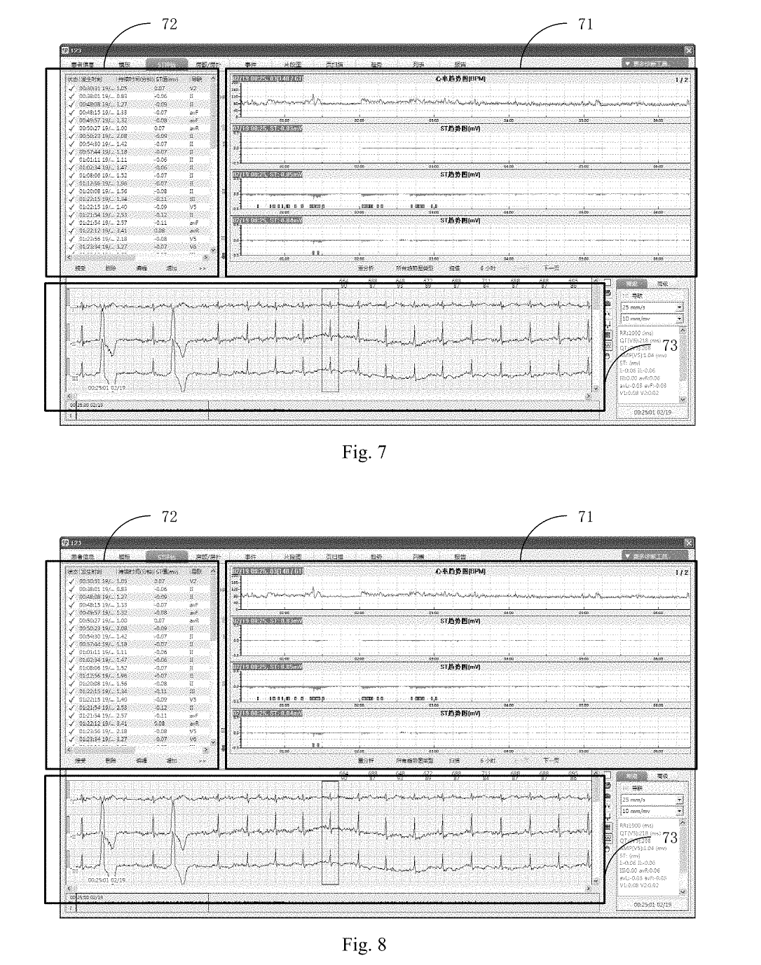

As one embodiment of the present invention, as shown in FIG. 7, when the amplitude trend graph 71 of the ST segment, the display area 72 of the ST event list, and the waveform area 73 of the ECG are simultaneously displayed on the screen, the above three parts of display areas are displayed on the same time axis. Therefore, if a selection event occurring in any one of the above three display areas is detected, the other two display areas may simultaneously jump to the display content at the same point in time as the point in time pointed by the selection event. For example, when the user selects one of the ST events in the display area of the ST event list, the amplitude trend graph of the ST segment is automatically positioned to the start time of the ST event, and the waveform area of the ECG is also automatically positioned to the start time of the ST event. When the user clicks on the amplitude trend graph of the ST segment at any point in time, the waveform area of the ECG is automatically positioned to the point in time. When the click position comprises an ST event, the display area of the ST event list is automatically positioned to a record of the ST event.

The linkage function of the above different display areas can help the user to quickly and comprehensively grasp related information of the ST event. For example, when the user views the ST event in the display area of the ST event list, the system can automatically and quickly displays related information of the ST event in the amplitude trend graph of the ST segment and the waveform area of the ECG. Therefore, the time for the user to find the related information of the ST event in other display areas is saved, and the operational efficiency is improved.

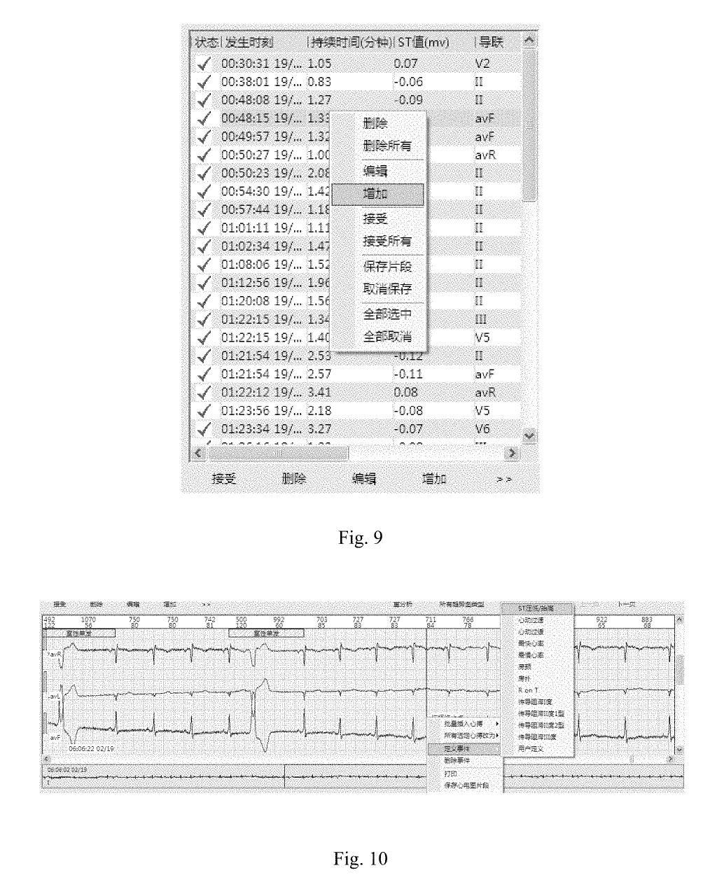

Additionally, as another embodiment of the present invention, when the amplitude trend graph of the ST segment, the display area of the ST event list, and the waveform area of the ECG are simultaneously displayed on the screen, the above three parts of display areas are displayed on the same time axis too. Therefore, if an editing operation for the ST event occurring in any of the above display areas is detected, the information of the ST event can be synchronously updated in the other two display areas according to the editing operation. Wherein the editing operation comprises, but is not limited to, addition, deletion or modification to the ST events. For example, interference or error may result in a false ST event, the user can modify or delete the ST event in any display area; or, for a software undetected ST event, the user can increase the ST event in any display area. Then each of the above editing operations will be synchronously updated in the other two display area. FIGS. 8 to 10 illustrate exemplary interface views of editing the ST event in the amplitude trend graph of the ST segment, the display area of the ST event list, and the waveform area of the ECG, respectively.

When an ST event is edited in the amplitude trend graph of the ST segment, an information window related to the ST event may be displayed in the amplitude trend graph of the ST segment. The information window comprises relevant edit information of the ST event, for example, a starting time of the ST even, a duration of the ST event, leads to detect the ST event, an amplitude of the ST event, and so on, so that the user can perform the editing operation of the ST event directly in the information window, and the convenience of operating the ST event is improved.

Additionally, in the display area of the ST event list, an editing operation of an ST event is marked. For example, when it is detected that the ST event is deleted, the ST event in the display area of the ST event list is cross-marked; when it is detected that the ST event is modified, the ST event in the display area of the ST event list is tick-marked.

It may be understood that, the sequence numbers of the steps in the above embodiments do not imply the order of execution, and the order in which the processes are executed should be determined by its function and inherent logic, and should not be construed as any limitation to implementation processes of the embodiments of the present invention.

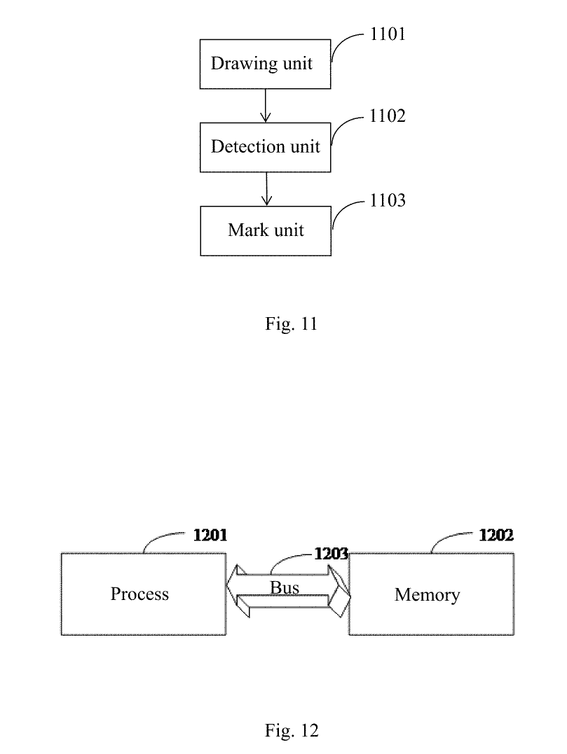

Corresponding to the method for presenting the ST event in the ECG described in the above embodiments, FIG. 11 illustrates a structural block diagram of a device for presenting an ST event in an ECG provided by an embodiment of the present invention. For the purpose of convenience of explanation, only the portions related to the present embodiment are shown.

Referring to FIG. 11, the device comprises:

a drawing unit 1101, configured for drawing an amplitude trend graph of an ST segment in the ECG;

a detection unit 1102, configured for detecting an ST event in the amplitude trend graph of the ST segment; and

a mark unit 1103, configured for marking the detected ST event in the amplitude trend graph of the ST segment.

Optionally, the drawing unit 1101 comprises:

a detection sub-unit, configured for performing a QRS complex detection for ECG data collected from each lead, and obtaining characteristic points of the QRS complex by the detection;

a first determining sub-unit, configured for determining the ST segment according to the characteristic points of the QRS complex; and

a first drawing sub-unit, configured for drawing the amplitude trend graph of the ST segment according to an amplitude of the ST segment.

Optionally, the drawing unit 1001 comprises:

a first dividing sub-unit, configured for dividing an amplitude value range of the ST segment into a plurality of numerical intervals, and setting a corresponding drawing color for each of the numerical intervals;

a second determining sub-unit, configured for determining a drawing color of an amplitude value corresponding to an point in time according to a numerical interval where the amplitude value corresponding to the point in time lies, for any point in time of the amplitude trend graph of the ST segment, and

a second drawing sub-unit, configured for drawing an amplitude value for each point in time of the amplitude trend graph of the ST segment, according to the determined drawing color.

Optionally, the detection unit 1102 comprises:

a second dividing sub-unit, configured for dividing an amplitude value range of the ST segment in a period of time corresponding to the ST event into a plurality of numerical intervals, and setting a corresponding mark format for each of the numerical intervals; and

a mark sub-unit, configured for determining a mark format corresponding to the ST event according to a numerical interval where an amplitude value of the ST event lies, and marking the ST event.

Optionally, the device further comprises:

a display unit, configured for, when it is detected that a mark of any ST event in the amplitude trend graph of the ST segment is triggered, displaying information of the ST event in a display area of an ST event list which is displayed in the same screen as the amplitude trend graph of the ST segment, and performing a display content jumping in an ECG waveform area which is displayed in the same screen as the amplitude trend graph of the ST segment, thereby displaying heartbeat data at the same time as a start position of the ST event in the ECG waveform area.

Optionally, the device further comprises:

a linkage unit, configured for, if a selection event occurring in any one of the amplitude trend graph of the ST segment, the display area of the ST event list, and the waveform area of the ECG is detected, the other two of the amplitude trend graph of the ST segment, the display area of the ST event list, and the waveform area of the ECG simultaneously jumping to the display content at the same point in time as the point in time pointed by the selection event.

Optionally, the device further comprises:

a synchronization updating unit, configured for, if an editing operation for an ST event occurring in any one of the amplitude trend graph of the ST segment, the display area of the ST event list, and the waveform area of the ECG is detected, information of the ST event being synchronously updated according to the editing operation in the other two of the amplitude trend graph of the ST segment, the display area of the ST event list, and the waveform area of the ECG.

Referring to FIG. 12, the device comprises a processor 1201, a memory 1202, and a bus 1203.

Wherein, the processor 1201 and the memory 1202 are communicated with each other through the bus 1203;

The memory 1202, is configured for storing programs.

The processor 1201 is configured for executing the programs stored in the memory 1202, when the programs are executed, configured for:

drawing an amplitude trend graph of an ST segment in the ECG; detecting an ST event in the amplitude trend graph of the ST segment; and

marking the detected ST event in the amplitude trend graph of the ST segment.

Optionally, the processor 1201 is specifically configured for:

performing a QRS complex detection for ECG data collected from each lead, and obtaining characteristic points of the QRS complex by the detection;

determining the ST segment according to the characteristic points of the QRS complex; and

drawing the amplitude trend graph of the ST segment according to an amplitude of the ST segment.

Optionally, the processor 1201 is specifically configured for:

dividing an amplitude value range of the ST segment into a plurality of numerical intervals, and setting a corresponding drawing color for each of the numerical intervals:

for any point in time of the amplitude trend graph of the ST segment, determining a drawing color of an amplitude value corresponding to the point in time according to a numerical interval where the amplitude value corresponding to the point in time lies; and

drawing an amplitude value for each point in time of the amplitude trend graph of the ST segment, according to the determined drawing color.

Optionally, the processor 1201 is specifically configured for:

dividing an amplitude value range of the ST segment in a period of time corresponding to the ST event into a plurality of numerical intervals, and setting a corresponding mark format for each of the numerical intervals; and

for any ST event, determining a mark format corresponding to the ST event according to a numerical interval where an amplitude value of the ST event lies, and marking the ST event.

Optionally, the processor 1201 is specifically configured for:

when it is detected that a mark of any ST event in the amplitude trend graph of the ST segment is triggered, displaying information of the ST event in a display area of an ST event list which is displayed in the same screen as the amplitude trend graph of the ST segment, and performing a display content jumping in an ECG waveform area which is displayed in the same screen as the amplitude trend graph of the ST segment, thereby displaying heartbeat data at the same time as a start position of the ST event in the ECG waveform area.

Optionally, the processor 1201 is specifically configured for:

if a selection event occurring in any one of the amplitude trend graph of the ST segment, the display area of the ST event list, and the waveform area of the ECG is detected, the other two of the amplitude trend graph of the ST segment, the display area of the ST event list, and the waveform area of the ECG simultaneously jumping to the display content at the same point in time as the point in time pointed by the selection event.

Optionally, the processor 1201 is specifically configured for:

if an editing operation for an ST event occurring in any one of the amplitude trend graph of the ST segment, the display area of the ST event list, and the waveform area of the ECG is detected, information of the ST event being synchronously updated according to the editing operation in the other two of the amplitude trend graph of the ST segment, the display area of the ST event list, and the waveform area of the ECG.

Those of ordinary skill in the art may clearly understand that, for the purposes of convenience and conciseness of the description, the foregoing functional units and modules are merely illustrated. In practical applications, the functions described above may be performed by different functional units and modules, that is, the internal structure of the device is divided into different functional units or modules to perform all or part of the functions described above. The functional units and modules in the embodiments may be integrated in one processing unit, or each unit and may be presented separately and physically, or two or more units may be integrated in one processing unit. The above integrated unit may be implemented in hardware form, and may also be implemented in the form of software functional unit. Additionally, the specific names of the functional units and modules are only intended to be distinguished from each other easily, but not to limit the protection scope of the present invention. The specific operations of the units and modules in the above-mentioned system can be referred to the corresponding processes in the aforementioned embodiments of the method, and will not be described here.

Those of ordinary skill in the art will recognize that, the units and algorithm steps of the various embodiments described above, may be implemented by electronic hardware, or by a combination of computer software and electronic hardware. Whether these functions are implemented by hardware or software depends on the specific application and design constraints of the solution. A skilled artisan may use different methods to implement the described functions for each particular application, but such implementations are not to be considered as departing from the scope of the present invention.

In the embodiments provided by the present invention, it is may be understood that, the disclosed apparatus and method may be implemented in other manners. For example, the system embodiments described above are merely illustrative, for example, the partitioning of the modules or units is only one logical functional partitioning, and the actual implementation may have additional partitioning method, such as multiple units or components may be integrated into another system, or some feature may be omitted, or not performed. Alternatively, the illustrated or discussed mutual coupling or direct coupling or communication connection may be an indirect coupling or a communication connection through interfaces, devices, or units, and may be in electrical, mechanical, or other forms.

The elements described as separate components may be or not be physically separated, and the elements described as display units may or may not be physical units, that is, may be located in one place, or may be distributed over a plurality of network elements. Some or all of the elements may be selected according to actual needs to realize the object of the present embodiment.

Additionally, the functional units in the various embodiments of the present invention may be integrated in one processing unit, or each unit may be physically present alone, or two or more units may be integrated in one unit. The above-mentioned integrated unit can be implemented in the form of hardware or a software functional unit.

if the integrated unit is implemented in the form of a software functional unit, and sold or used as a standalone product, the integrated unit may be stored in a computer-readable storage medium. Based on this understanding, essence of the technical solution of the embodiments of the present invention, or the part contributing to the prior art, or all or part of the technical solution, can be embodied in the form of a computer software product. The computer software product is stored in a storage media, and the computer software product comprises a number of instructions to enable a computer device (which may be a personal computer, server, or network device, etc.) or processor to perform all or part of the steps of the method described in the various embodiments of the present invention. The storage medium comprises a USB disk, a removable hard disk, a read-only memory (ROM), a random access memory (RAM), a magnetic disk, an optical disk, and other medium which can store program code.

The embodiments described above are merely used to illustrate the technical solutions of the present invention, and are not to be construed as limitations. While the invention has been described in detail with reference to the foregoing embodiments, it will be understood by those skilled in the art that, the technical solutions described in the foregoing embodiments of the present invention may be modified, or some of the technical features can be equivalently substituted, and these modifications and substitutions do not make corresponding technical solutions depart from the spirit and scope of the technical solutions of the embodiments of the present invention, and should be included in the protection scope of the present invention.

What is claimed is:

1. A method for presenting an ST event in an electrocardiogram (ECG), comprising:
drawing an amplitude trend graph of an ST segment in the ECG;
detecting an ST event in the amplitude trend graph of the ST segment; and
marking the detected ST event in the amplitude trend graph of the ST segment.

2. The method as claimed in claim 1, wherein drawing an amplitude trend graph of an ST segment in the ECG comprises:
performing a QRS complex detection for ECG data collected from each lead, and obtaining characteristic points of the QRS complex by the detection;
determining the ST segment according to the characteristic points of the QRS complex; and
drawing the amplitude trend graph of the ST segment according to an amplitude of the ST segment.

3. The method as claimed in claim 1, wherein drawing an amplitude trend graph of an ST segment in the ECG comprises:
dividing an amplitude value range of the ST segment into a plurality of numerical intervals, and setting a corresponding drawing color for each of the numerical intervals;
for any point in time of the amplitude trend graph of the ST segment, determining a drawing color of an amplitude value corresponding to the point in time according to a numerical interval where the amplitude value corresponding to the point in time lies; and
drawing an amplitude value for each point in time of the amplitude trend graph of the ST segment, according to the determined drawing color.

4. The method as claimed in claim 1, wherein marking the detected ST event in the amplitude trend graph of the ST segment comprises:
dividing an amplitude value range of the ST segment in a period of time corresponding to the ST event into a plurality of numerical intervals, and setting a corresponding mark format for each of the numerical intervals; and
for any ST event, determining a mark format corresponding to the ST event according to a numerical interval where an amplitude value of the ST event lies, and marking the ST event.

5. The method as claimed in claim 1, wherein, the method further comprises:
when it is detected that a mark of any ST event in the amplitude trend graph of the ST segment is triggered, displaying information of the ST event in a display area of an ST event list which is displayed in the same screen as the amplitude trend graph of the ST segment, and performing a display content jumping in an ECG waveform area which is displayed in the same screen as the amplitude trend graph of the ST segment, thereby displaying heartbeat data at the same time as a start position of the ST event in the ECG waveform area.

6. The method as claimed in claim 5, wherein, the method further comprises:
if a selection event occurring in any one of the amplitude trend graph of the ST segment, the display area of the ST event list, and the waveform area of the ECG is detected, the other two of the amplitude trend graph of the ST segment, the display area of the ST event list, and the waveform area of the ECG simultaneously jumping to the display content at the same point in time as the point in time pointed by the selection event.

7. The method as claimed in claim 5, wherein, the method further comprises:
if an editing operation for an ST event occurring in any one of the amplitude trend graph of the ST segment, the display area of the ST event list, and the waveform area of the ECG is detected, information of the ST event being synchronously updated according to the editing operation in the other two of the amplitude trend graph of the ST segment, the display area of the ST event list, and the waveform area of the ECG.

8. A device for presenting an ST event in an ECG comprising:
a drawing unit, configured for drawing an amplitude trend graph of an ST segment in the ECG;
a detection unit, configured for detecting an ST event in the amplitude trend graph of the ST segment; and
a mark unit, configured for marking the detected ST event in the amplitude trend graph of the ST segment.

9. The device as claimed in claim 8, wherein, the device further comprises:
a display unit, configured for, when it is detected that a mark of any ST event in the amplitude trend graph of the ST segment is triggered, displaying information of the ST event in a display area of an ST event list which is displayed in the same screen as the amplitude trend graph of the ST segment, and performing a display content jumping in an ECG waveform area which is displayed in the same screen as the amplitude trend graph of the ST segment, thereby displaying heartbeat data at the same time as a start position of the ST event in the ECG waveform area.

10. The device as claimed in claim 9, wherein, the device further comprises:
a linkage unit, configured for, if a selection event occurring in any one of the amplitude trend graph of the ST segment, the display area of the ST event list, and the waveform area of the ECG is detected, the other two of the amplitude trend graph of the ST segment, the display area of the ST event list, and the waveform area of the ECG simultaneously jumping to the display content at the same point in time as the point in time pointed by the selection event.

11. A device for presenting an ST event in an ECG, comprising a processor, a memory, and a bus;
the processor and the memory communicated with each other through the bus;
the memory, configured for storing programs; and
the processor configured for executing the programs stored in the memory, when the programs executed, configured for:
drawing an amplitude trend graph of an ST segment in the ECG;
detecting an ST event in the amplitude trend graph of the ST segment; and
marking the detected ST event in the amplitude trend graph of the ST segment.

12. The device as claimed in claim 11, wherein the processor is specifically configured for:
performing a QRS complex detection for ECG data collected from each lead, and obtaining characteristic points of the QRS complex by the detection;
determining the ST segment according to the characteristic points of the QRS complex; and
drawing the amplitude trend graph of the ST segment according to an amplitude of the ST segment.

13. The device as claimed in claim 11, wherein the processor is specifically configured for:
dividing an amplitude value range of the ST segment into a plurality of numerical intervals, and setting a corresponding drawing color for each of the numerical intervals:
for any point in time of the amplitude trend graph of the ST segment, determining a drawing color of an amplitude value corresponding to the point in time according to a numerical interval where the amplitude value corresponding to the point in time lies; and
drawing an amplitude value for each point in time of the amplitude trend graph of the ST segment, according to the determined drawing color.

14. The device as claimed in claim 11, wherein the processor is specifically configured for:
dividing an amplitude value range of the ST segment in a period of time corresponding to the ST event into a plurality of numerical intervals, and setting a corresponding mark format for each of the numerical intervals; and
for any ST event, determining a mark format corresponding to the ST event according to a numerical interval where an amplitude value of the ST event lies, and marking the ST event.

15. The device as claimed in claim 11, wherein the processor is specifically configured for:
when it is detected that a mark of any ST event in the amplitude trend graph of the ST segment is triggered, displaying information of the ST event in a display area of an ST event list which is displayed in the same screen as the amplitude trend graph of the ST segment, and performing a display content jumping in an ECG waveform area which is displayed in the same screen as the amplitude trend graph of the ST segment, thereby displaying heartbeat data at the same time as a start position of the ST event in the ECG waveform area.

16. The device as claimed in claim 15, wherein the processor is specifically configured for:
if a selection event occurring in any one of the amplitude trend graph of the ST segment, the display area of the ST event list, and the waveform area of the ECG is detected, the other two of the amplitude trend graph of the ST segment, the display area of the ST event list, and the waveform area of the ECG simultaneously jumping to the display content at the same point in time as the point in time pointed by the selection event.

17. The device as claimed in claim 15, wherein the processor is specifically configured for:
if an editing operation for an ST event occurring in any one of the amplitude trend graph of the ST segment, the display area of the ST event list, and the waveform area of the ECG is detected, information of the ST event being synchronously updated according to the editing operation in the other two of the amplitude trend graph of the ST segment, the display area of the ST event list, and the waveform area of the ECG
